(19) United States
(12) Patent Application Publication
(10) Pub. No.: US 2007/0161896 A1
(43) Pub. Date: Jul. 12, 2007

(54) Capacitive micromachined ultrasonic transducer (cMUT) and its production method

(75) Inventors: Hideo Adachi, Iruma (JP); Katsuhiro Wakabayashi, Tokyo (JP); Shinji Yasunaga, Tokyo (JP); Kiyoshi Nemoto, Tokyo (JP); Miyuki Murakami, Tokyo (JP)

Correspondence Address: Thomas Spinelli;Scully, Scott, Murphy & Presser, 400 Garden City Plaza, Garden City, NY, 11530, US

(73) Assignee: OLYMPUS CORPORATION, Tokyo (JP)

(21) Appl. No.: 11/702,277

(22) Filed: Feb. 5, 2007

Related U.S. Application Data
(63) Continuation of application No. PCT/JP05/13190, filed on Jul. 15, 2005.

(30) Foreign Application Priority Data
Aug. 5, 2004 (JP) 2004-229379

(51) Int. Cl. A61B 8/00

(52) U.S. Cl. 600/437

(57) ABSTRACT

A capacitive micromachined ultrasonic transducer (cMUT) at least including a silicon substrate, a bottom electrode mounted onto the silicon substrate, a upper electrode mounted facing the bottom electrode and apart therefrom by a predetermined cavity, and a membrane supporting the upper electrode, wherein a part of the aforementioned cMUT is charged.

### CROSS REFERENCE TO RELATED APPLICATIONS

This is a Continuation Application of PCT Application No. PCT/JP2005/013190 filed, filed Jul. 15, 2005, which was not published under PCT Article 21(2) in English.

This application is based upon and claims the benefit of priority from the prior Japanese Patent Application No. 2004-229379 filed in Japan on Aug. 5, 2004, the entire contents of which are incorporated herein by reference.

### BACKGROUND OF THE INVENTION

1. Field of the Invention

The present invention relates to a capacitive micromachined ultrasonic transducer (cMUT) produced by processing a silicon substrate by using a silicon micromachining technique.

2. Description of the Related Art

An ultrasonic diagnostic method is widely used for diagnosis by transmitting ultrasound waves into an abdomen and imaging an internal state of body from an echo signal of the waves. Possible equipment used for ultrasonic diagnostic method is an ultrasonic endoscope, which is equipped with an ultrasonic transducer at the tip of an insertion part that is inserted into an abdomen. The ultrasonic transducer is configured to transmit ultrasound waves into the abdomen, by converting an electric signal to an ultrasound wave, and receive ultrasound waves reflected by the abdomen, by converting them to an electric signal.

Although some conventional ultrasonic transducers use a ceramic piezo-electric material PZT (i.e., lead zirconate titanate) as a piezo-electric device that converts the electric signal to ultrasound waves, what is attracting attention is a capacitive ultrasonic transducer (e.g., a Capacitive Micromachined Ultrasonic Transducer (abbreviated as “cMUT” hereinafter)) made by processing a silicon semiconductor substrate by employing a silicon micromachining technique. This type of device is typically referred to as a micromachine or otherwise known as a Micro Electric-Mechanical System (MEMS), which is for example, an ultra miniature electro-mechanical complex.

A MEMS device, for example, being formed with a miniature structure on a substrate such as silicon substrate or glass substrate, is a device that electronically and mechanically combines a drive body for outputting a mechanical driving force, a driver mechanism for driving the drive body, and a semiconductor integrated circuit for controlling the driver mechanism. A fundamental characteristic of the MEMS device is that the drive body comprises a mechanical structure built in as a part of the device, with a drive of the drive body being electrically carried out by applying coulomb attraction force between electrodes.

A c-MUT is a device having two flat electrodes facing each other, having a cavity between the two flat electrodes and generating an ultrasound wave by a membrane vibration, which includes one of the aforementioned two flat electrodes, harmonically vibrating when an radio frequency (RF) signal is applied to the membrane by superimposing with a direct current (DC) bias (e.g., refer to a patent document 1).

FIG. 1(a) shows a cell structure of a conventional cMUT 310. Referring to FIG. 1(a), a bottom electrode 322 is formed on the surface (i.e., in the back) of a silicon substrate 312, and a membrane 314 is supported by membrane support parts 316. An upper electrode 320 is formed on the membrane 314, and a cavity 318 is formed within the above described components.

FIG. 1(b) is an operation description diagram of the configuration shown by FIG. 1A. The bottom electrode 322 is grounded, and an RF signal for generating an ultrasound wave is applied through a terminal 326 to the upper electrode 320 by superimposing a DC bias voltage V_{B} through a terminal 324. As such, a DC bias is required for both transmitting and receiving an ultrasound wave.

As shown in FIG. 1(b), the DC bias voltage V_{B} superimposed with the RF signal through the terminal 326 is required for a transmission and a reception.

FIG. 2 shows a time chart of a conventional drive voltage, with FIG. 2(a) showing a time chart of a drive voltage of an RF signal, while FIG. 2(b) shows that of a drive voltage of a DC bias voltage V_{B}. An ultrasonic diagnosis usually obtains a diagnostic image by converting a pulse echo signal, which is acquired by transmitting and receiving an RF pulse signal, into an image signal. However, referring to FIG. 2(a), a reception period T_{b} for receiving a pulse echo signal is long, for example, between 0.05 and 1.0 milliseconds, as compared to a transmission pulse signal transmission period Ta, for example, that is bellow several microseconds. If a time period for a transmission pulse signal transmission was a period of only several microseconds, the effective voltage of the RF pulse signal would be very small even though a peak voltage of transmission pulse signal is hundreds of volts. However, referring to FIG. 2(b), a continuous application of a DC voltage of hundreds of volts DC force entire reception period creates an excessive effective value of the drive voltage, and hence is undesirable.

In consideration of the above described problem, the present invention provides a cMUT driven only by an RF pulse signal without a DC bias voltage.

Patent document 1: Laid-Open Japanese Patent Application Publication No. 2004-503313

### SUMMARY OF THE INVENTION

A cMUT according to the present invention is one at least including a silicon substrate, a bottom electrode mounted onto the silicon substrate, a upper electrode mounted facing the bottom electrode and apart therefrom by a predetermined cavity, and a membrane supporting the upper electrode, wherein a part of the aforementioned cMUT is charged.

Also according to the present invention, a production method for a cMUT at least including a silicon substrate, a bottom electrode mounted onto the silicon substrate, a upper electrode mounted facing the bottom electrode and apart therefrom by a predetermined cavity, and a membrane supporting the upper electrode comprises the following processes: forming the bottom electrode on the silicon substrate; forming a dielectric film on a surface of the bottom electrode; carrying out a corona charging treatment, with the bottom electrode being grounded, such that the dielectric film has a surface potential; forming the membrane and a mounting part for supporting therefor; and forming the upper electrode on the membrane.

Also according to the present invention, a production method for a cMUT at least including a silicon substrate, a bottom electrode mounted onto the silicon substrate, a upper electrode mounted facing the bottom electrode and apart therefrom by a predetermined cavity, and a membrane supporting the upper electrode, comprising: a process for forming the bottom electrode on the silicon substrate; a first dielectric film forming process for forming a dielectric film on a surface of the bottom electrode; a first charging process for applying a corona charging treatment, with the bottom electrode being grounded, so that the dielectric film formed by the first dielectric film forming process has a surface potential; a process for forming the membrane and a support part for supporting the membrane; a process for forming the upper electrode on the membrane; a second dielectric film forming process for forming a dielectric film on the upper electrode; and a second charging process for applying a corona charging treatment, with the upper electrode being grounded, so that the dielectric film formed by the second dielectric film forming process has a surface potential.

Also according to the present invention, a production method for a cMUT at least including a silicon substrate, a bottom electrode mounted onto the silicon substrate, a upper electrode mounted facing the bottom electrode and apart therefrom by a predetermined cavity, and a membrane supporting the upper electrode, that comprises: a first structure forming process for generating a first structure by a process for forming the bottom electrode on a first silicon substrates, a first dielectric film forming process for forming a dielectric film on a surface of the bottom electrode, a first charging process for applying a corona charging treatment, with the bottom electrode being grounded, so that a dielectric film formed by the first dielectric film forming process has a surface potential, and a process for forming a support part in order to support the membrane; a second structure forming process for generating a second structure by a second charging process for applying a corona charging treatment, with a second silicon substrate whose surface has been applied by an oxidization treatment being grounded, so that the oxidized film on the surface has a surface potential, and a process for forming the upper electrode on the oxidized film having a surface potential by the second charging treatment; and a process for connecting between the first structure generated by the first structure forming process and the second structure generated by the second structure forming process.

Also according to the present invention, a production method for a cMUT at least including a silicon substrate, a bottom electrode mounted onto the silicon substrate, a upper electrode mounted facing the bottom electrode and apart therefrom by a predetermined cavity, and a membrane supporting the upper electrode, that comprises: a first structure forming process for generating a first structure by a process for forming a bottom electrode on a first silicon substrates, a first dielectric film forming process for forming a dielectric film on a surface of the bottom electrode, a first charging process for applying a corona charging treatment, with the bottom electrode being grounded, so that a dielectric film formed by the first dielectric film forming process has a surface potential, and a process for forming a support part in order to support the membrane; a second structure forming process for generating a second structure by a second charging process for applying a corona charging treatment, with a second silicon substrate whose surface has been applied by an oxidization treatment being grounded, so that the oxidized film on the surface has a surface potential, a process for forming the upper electrode on the oxidized film having a surface potential by the second charging treatment, a process for forming a dielectric film having a high dielectric constant on a surface of the upper electrode, and a third charging process for applying a corona charging treatment, with the upper electrode being grounded, so that the dielectric film having a high dielectric constant has a surface potential; and a process for connecting between the first structure generated by the first structure forming process and the second structure generated by the second structure forming process.

### BRIEF DESCRIPTION OF DRAWINGS

FIG. 1 is a description diagram relating to a conventional cMUT;

FIG. 2 is a diagram showing a time chart of a conventional drive voltage;

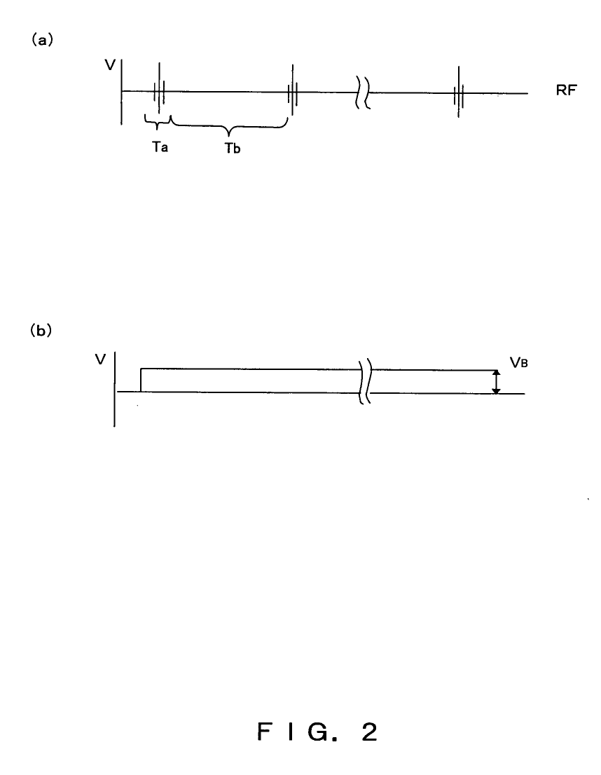

FIG. 3 is an overall cross-section diagram of a fundamental structure of a cMUT cell according to a first embodiment;

FIG. 4A is a diagram showing a production process of a cMUT 1 according to the first embodiment;

FIG. 4B is a diagram showing a detailed process of FIG. 4A (d);

FIG. 5 is a diagram for describing a corona discharge according to the first embodiment;

FIG. 6 is a diagram showing how a surface potential of a dielectric film changes with the number of elapsed days according to the first embodiment;

FIG. 7 is a diagram showing an effect of a presence or absence of a heat treatment after forming a dielectric film according to the first embodiment;

FIG. 8 is a diagram showing a result of a DC bias application test according to the first embodiment;

FIG. 9 is an overall cross-section diagram of a fundamental structure of a cMUT cell according to a second embodiment;

FIG. 10A is a diagram showing a production process of a cMUT 51 according to the second embodiment (part 1);

FIG. 10B is a diagram showing a production process of a cMUT 51 according to the second embodiment (part 2);

FIG. 11 is a diagram for describing a corona discharge according to the second embodiment;

FIG. 12 is an overall cross-section diagram of a fundamental structure of a cMUT cell according to a third embodiment;

FIG. 13A is a diagram showing a production process of a cMUT 71 according to the third embodiment (part 1);

FIG. 13B is a diagram showing a production process of a cMUT 71 according to the third embodiment (part 2);

FIG. 13C is a diagram showing a production process of a cMUT 71 according to the third embodiment (part 3);

FIG. 14 is a diagram for describing a corona discharge according to the third embodiment;

FIG. 15 is an overall cross-section diagram of a fundamental structure of a cMUT cell according to a fourth embodiment;

FIG. 16A is a diagram showing a production process of a cMUT 91 according to the fourth embodiment (part 1);

FIG. 16B is a diagram showing a production process of a cMUT 91 according to the fourth embodiment (part 2);

FIG. 16C is a diagram showing a production process of a cMUT 91 according to the fourth embodiment (part 3);

FIG. 17 is a diagram for describing a corona discharge according to the fourth embodiment;

FIG. 18 is an overall cross-section diagram of a fundamental structure of a cMUT cell according to a fifth embodiment;

FIG. 19 is a diagram showing a production process of a cMUT 111 according to the fifth embodiment;

FIG. 20 is an overall cross-section diagram of a fundamental structure of a cMUT cell according to a sixth embodiment;

FIG. 21 is a diagram for describing a corona discharge according to the sixth embodiment;

FIG. 22A is a diagram showing a production process of a cMUT according to a seventh embodiment (part 1);

FIG. 22B is a diagram showing a production process of a cMUT according to the seventh embodiment (part 2);

FIG. 22C is a diagram showing a production process of a cMUT according to the seventh embodiment (part 3); and

FIG. 22D is a diagram showing a production process of a cMUT according to the seventh embodiment (part 4).

### DESCRIPTION OF THE PREFERRED EMBODIMENTS

The present invention is based on forming a dielectric film (i.e., an insulator film) on a predetermined part of a cMUT and charging the dielectric film. This creates a similar effect to a cMUT as does applying a DC bias voltage. Therefore, the present invention makes it possible to drive the cMUT with only an RF signal without applying a DC bias voltage. Various cMUTs may be produced by varying different characteristics of the dielectric film, for example, such as position, material, or composition of a dielectric film for charging. The following are the preferred embodiments according to the present embodiment.

### First Embodiment

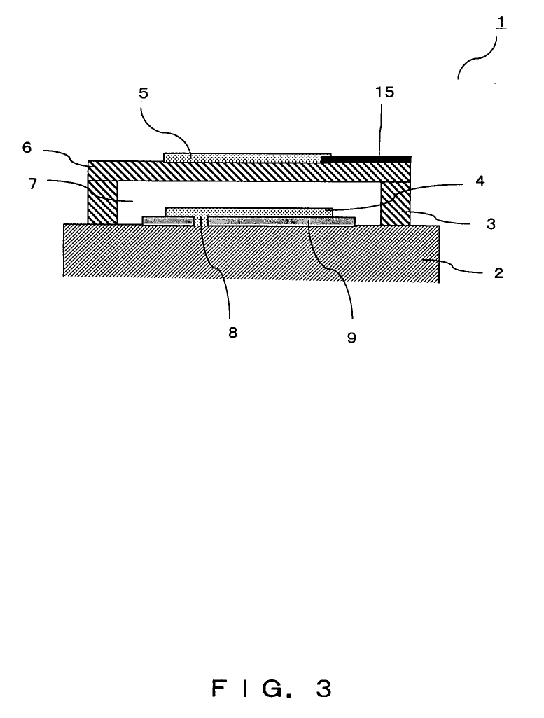

FIG. 3 is an overall cross-section diagram of a fundamental structure of a cMUT cell according to the present embodiment. The cMUT cell comprises a silicon substrate 2, a dielectric film 9, a bottom electrode 4, a membrane 6, a cavity 7, an upper electrode 5, a Via hole 8, and a wiring film 15, wherein, the cMUT 1 comprises a plurality of cMUT cells.

The membrane 6 is an vibrating film with edge parts fixed by membrane support parts 3. An upper surface of the membrane 6 is equipped with the upper electrode 5. A dielectric film 9 (e.g., SiO₂) is formed on a front surface (i.e., the bottom part of a concave part) of the silicon substrate 2 between the membrane support parts 3 on which the bottom electrode 4 is equipped. The bottom electrode 4 is electrically connected to the silicon substrate 2 through the Via hole 8, and a conductor of the same material as the bottom electrode 4 is further laid thereunder. A patterned interconnection film 15 is connected to the upper electrode 5 and is drawn out to the outside of the cells constituting the cMUT 1. The wiring film 15 is a metallic film for inputting and outputting an electric signal to and from the upper electrode 5.

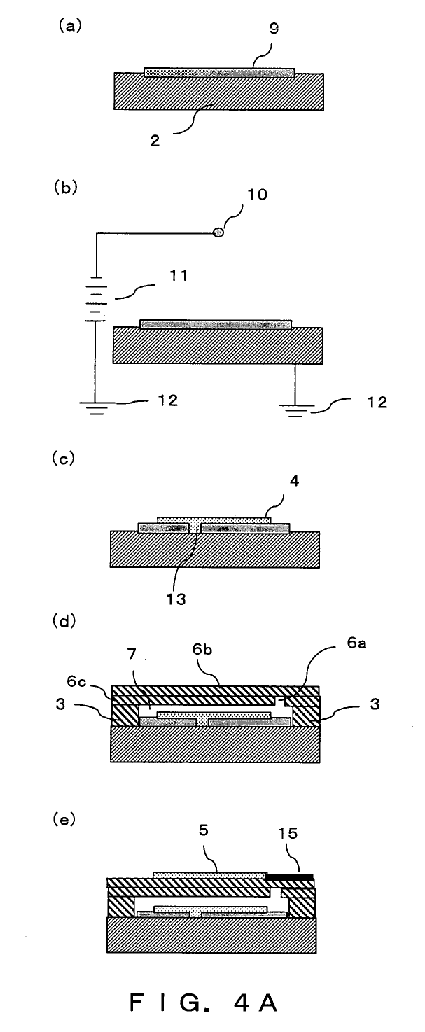

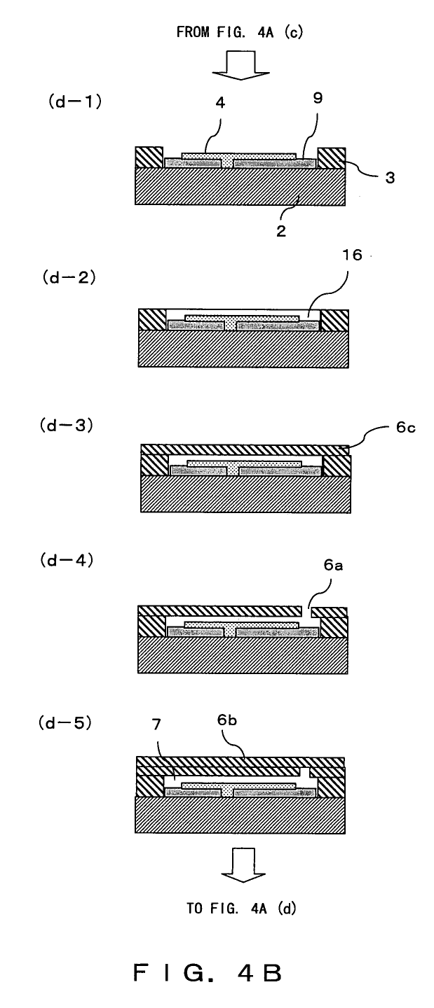

Note that the air cavity 7 is defined as a space surrounded by the membrane 6, membrane support parts 3, bottom electrode 4 and dielectric film 9. The membrane 6 may also be a plurality of membrane films in terms of the production process as described later by referring to FIG. 4.

Describing an operation of the cMUT 1, an application of a voltage to a pair of electrodes, for example, the upper electrode 5 and bottom electrode 4, causes the two electrodes to attract each other, and return to the original state when the voltage is zero. As a result of the membrane 6 vibration by the vibrating operation, ultrasound waves are generated and emitted in an upward direction of the upper electrode 5.

FIG. 4, i.e. FIGS. 4A and 4B, are diagrams each showing a production process of the cMUT 1 according to the present embodiment. FIG. 4B is a diagram showing details of the process used to make the device shown in FIG. 4A (d). First, a silicon dioxide film 9 is formed (i.e., forming an SiO₂ film) by means of thermal oxidization, RF magnetron sputtering, plasma chemical vapor deposition (CVD), a vacuum arc plasma method, or a sol-gel method, for example, on a low resistance silicon substrate 2. Then the first heat treatment is applied in the air or a nitrogen environment at a temperature between 300 and 800° C. (refer to FIG. 4A (a)).

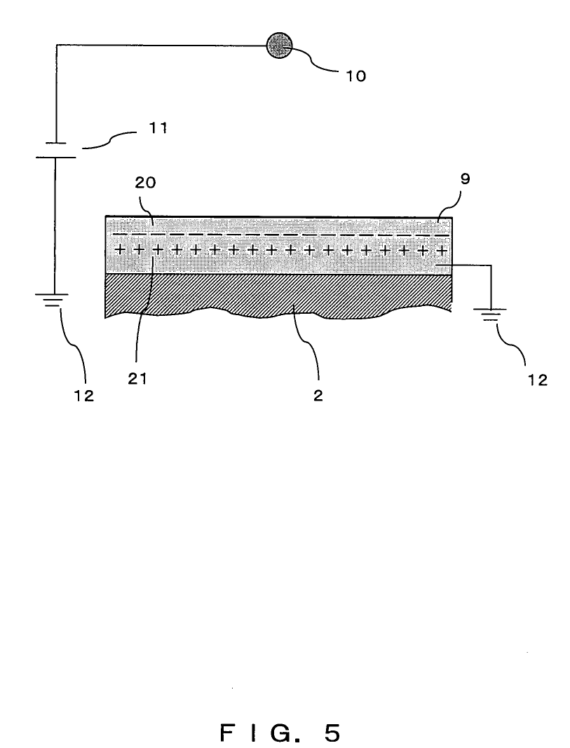

Next, the silicon substrate 2 is grounded (at the numeral 12), and a high DC voltage 11 of several kilovolts is applied between the silicon substrate 2 and a wire form electrode 10, there by making the latter emit a corona discharge and thereby charge the silicon dioxide film 9 (for example, a process for turning a material to an electret, referred to as “electretization” hereinafter) (refer to FIG. 4A (b) and FIG. 5). The top surface of the silicon dioxide film 9 is charged with a negative charge, while the silicon substrate side of the silicon dioxide film 9 is charged with a positive charge. This is described in detail by referring to FIG. 5.

FIG. 5 is a diagram for describing a corona discharge (as a process for turning a material into an electret, or “electretization”) according to the present embodiment). In FIG. 5, the wire form electrode 10 extends in the vertical direction relative to the drawing. A negative side of the high DC voltage 11 is connected to the electrode 10, while the positive side is grounded at the numeral 12. The electrode 10 is placed above the silicon substrate 2 on which the silicon dioxide film 9 is formed.

Now, as a high DC voltage 11 of several kilovolts is applied to cause a corona discharge, a negative charge is discharged from the electrode 10, thereby charging the top surface of the silicon dioxide film 9 with a negative charge (the numeral 20) and the silicon substrate side with a positive charge (the numeral 21).

The charge capacity can be adjusted by using different material, or changing the composition ratio, for example, of the dielectric film. The configuration is such that the dielectric film is charged in the direction to increase the field strength between the upper electrode 5 and bottom electrode 4 of the device shown in FIG. 3. Assuming that the upper electrode 5 is a negative pole and the bottom electrode 4 a positive pole for example, the electric field is generated upward from the bottom electrode 4 to upper electrode 5. Accordingly the top surface of the silicon dioxide film 9 is charged with a negative charge (the numeral 20), while the silicon substrate side of the silicon dioxide film 9 is charged with a positive charge (the numeral 21), thereby allowing forcee charging of the dielectric film in order to line up with the direction of the increasing field. Incidentally, the present embodiment calls such process for making a dielectric film charged by a corona discharge as “electretization” process. Note that a corona discharge may be carried out by reciprocating the substrate side in the lateral direction of the drawing in order to make it evenly charged. Or, for example, a grid electrode may be placed between the electrode and a charge process target body, thereby improving a stability of a corona discharge condition. The next description is of FIG. 4A.

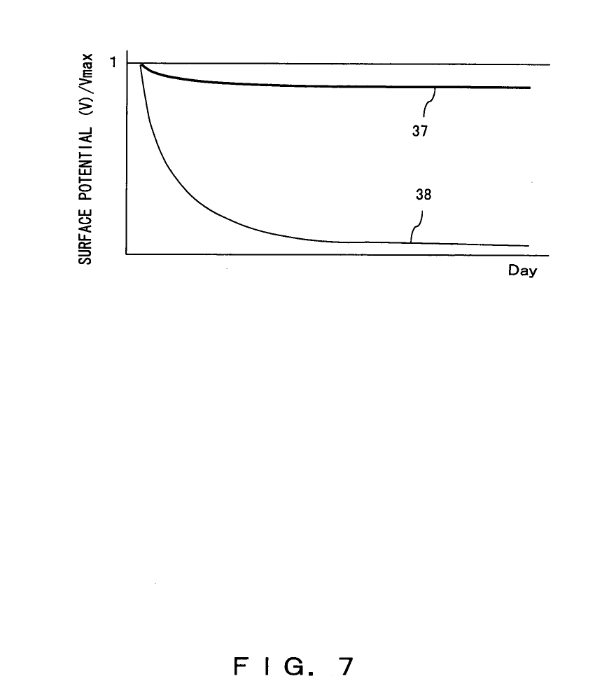

The next process described is for making a charge condition of the charged silicon dioxide film 9 (i.e., an aging treatment), for example, by using a heat treatment for one hour in the air at 150° C. (refer to FIG. 4A (b). A stability of a charge over time is important, and therefore the above mentioned heat treatment and aging treatment after the charge process are necessity for stabilization (to be described later by referring to FIG. 7).

As shown in FIG. 4A (c), the Via hole 13 is configured in the silicon dioxide film 9 and then the bottom electrode 4 made of gold (Au) or aluminum (Al) is formed (i.e., a bottom electrode filming). In this process, a bottom electrode 4 material, gold or aluminum for example, is accumulatively fills in the Via hole 13, resulting in forming a conductive path between the bottom electrode 4 and silicon substrate 2.

Referring to FIG. 4A (d), the air cavity 7 is formed by the following processes: a support part (SiNₓ film) forming, a sacrifice layer poly silicon film forming, a membrane film (SiNₓ film) forming, a sacrifice layer etching and an etching hole cover layer forming. Details of there processes are described by referring to FIG. 4B.

First, the membrane support parts 3 (e.g., Si₃N₄ film) are formed (refer to FIG. 4B (d-1)), followed by forming the membrane 6c made of Si₃N₄ and the air cavity 7 by means of a sacrifice layer etching, for example. More specifically, as shown in FIG. 4B (d-2) a sacrifice layer 16, which is sacrificed for forming a cavity part, for example, (i.e., a temporary layer which is later removed) is formed. The sacrifice layer 16 is formed by a material, for example, poly-silicon, which is easily removable by etching or another removal process.

Next, the membrane 6c constituting a membrane film is formed so as to cover the upper surface of the sacrifice layer 16 by using a membrane material (refer to FIG. 4B (d-3)). This is followed by removing the sacrifice layer 16 by etching, for example, and a second membrane film 6b is formed in order to cover a sacrifice layer material ejection hole 6a which was configured at the time of etching the sacrifice layer 16 (refer to FIG. 4B (d-5)). A material of the second membrane film may be the same as that of the membrane support parts 3 (e.g., Si₃N₄), however, other material may be used, such as silicon dioxide (SiO₂)

Finally, the upper electrode 5 and wiring film 15, made of gold (Au) or aluminum (Al), for example, are formed (refer to FIG. 4A (e)).

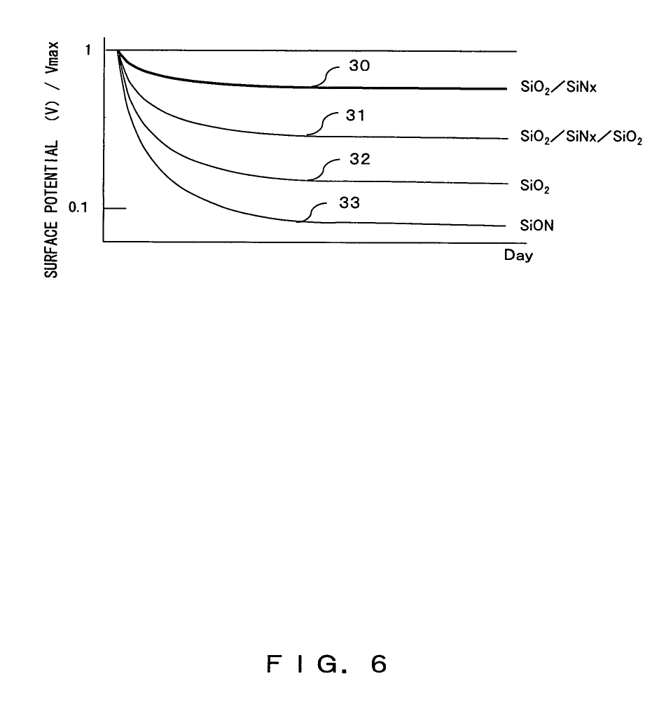

Note that the dielectric film 9 may use a silicon nitride film, or for example, a double layer made of SiO₂ and Si₃N₄ may be used (it is described later by referring to FIG. 6) in lieu of being limited by a silicon oxide film. Additionally, a dielectric film may use any appropriate material having a high dielectric constant such as barium titanate (BaTiO₃), strontium titanate (SrTiO₃), barium-strontium titanate, tantrum penta-oxide, niobium oxide-stabilized tantrum penta-oxide, aluminum oxide, or titanium dioxide (TiO₂), for example.

FIG. 6 is a diagram showing how a surface potential of a dielectric film changes with the number of elapsed days according to the present embodiment. A surface potential is a static voltage difference between the inside and outside of a focused system (e.g., a dielectric film). On a surface of a solid body, there are two phases contacting with each other (e.g., a solid body (i.e., a dielectric body) and a gas (i.e., air) ), in which state electrons, ions or dipoles, distribute unevenly to cause an electrical double layer. In addition, charges that are released into a solid body usually exist with various charge densities. In such a state (i.e., a system), the carrying of charged particles to the inside of a focused system from a distance, for example, an infinite distance, is similar to the process of letting a discharge current flow between both electrodes by a corona discharge. An amount of work required for carrying charged particles to the inside of the focused system from an infinite distance is called an electro chemical potential that is expressed by a sum of an amount of work W′ at the time of carrying a charge to the inside of a system and an amount of work W″ at the time of carrying a charged particle to the inside of a shell consisting only of an electrical double layer and a released charge. For example, it is similar to a shell that has no substance but is in a state of an electrical double layer and a released charge floating within a vacuum), assuming that there exists a system having neither the above described electrical double layer nor a released charge. The work amount W′ is a true interaction between the charged particle and focused system (e.g., a dielectric film), which is called a chemical potential and is a constant determined from the kind of material used and an arrayed state of a grid. Additionally, the work amount W″ is expressed by the product of a charge and a potential difference ψ_{α} (for example, the “internal potential” of the system) between the inside of a shell to which the charge is carried and the infinite distance. The internal potential can be further divided into a part χ_{α}, which is due to the electrical double-layer and a part ψ_{α}, which is due to the released charge. Between the two, the part χ_{α} due to the electrical double-layer is a static potential difference that is called a surface potential.

The diagram of FIG. 6 shows the case of forming various thin films on a silicon substrate and tracking how the surface potentials of the respective thin films change with an elapsed time. The curve 30 shows a surface potential change of a two-layer dielectric film constituted by SiO₂and SiNₓ. The curve 31 shows a surface potential change of a three-layer dielectric film constituted by SiO₂, SiNₓ and SiO₂. The curve 32 shows a surface potential change of a SiO₂ dielectric film. The curve 33 shows a surface potential change of an SiON dielectric film.

Although a static charge is stabilized a little by an aging treatment applied immediately after a charging process, the material of the dielectric film and a heat treatment application thereof influence problems of secular change over several years or more. FIG. 6 shows a comparable plot of decaying states of surface charge conditions with different types of materials and number of layers of dielectric films, showing that the decay of the single SiO_{2-α}Nα layer film (the curve 33) is the largest, while the decay characteristics improve going from the SiO₂ film (the curve 32), to three-layer film (the curve 31) consisting of SiO₂, SiNₓ and SiO₂, and then to two-layer film (the curve 30) consisting of SiO₂ and SiNₓ.

A dielectric film surface potential decay differs with film material and layer structure as described above. The smallest change of a surface potential is observed using the two-layer film (the curve 30), consisting of SiO₂ and SiNₓ (the curve 30). The surface potential's decay is far smaller as compared to the case of using a single layer of SiO₂.

FIG. 7 is a diagram showing an effect of the presence or absence of a heat treatment after forming a dielectric film, that is, tracking how a surface potential changes when applying a heat treatment and not applying a heat treatment after forming the film. The curve 37 shows a secular change curve of a surface potential when applying a heat treatment and an aging after forming the film. The curve 38 shows a secular change curve of a surface potential when applying neither a heat treatment nor an aging after forming the film.

From FIG. 7, applying a heat treatment and an aging after forming the film shows a smaller decay of the surface potential as compared to applying neither a heat treatment nor an aging. FIG. 4 shows the situation where a heat treatment is applied after forming the film. When all films are the same, when a heat treatment is not applied forcee SiO₂/SiNₓ, the layered film shows a large decay of a surface potential as shown by the curve 38.

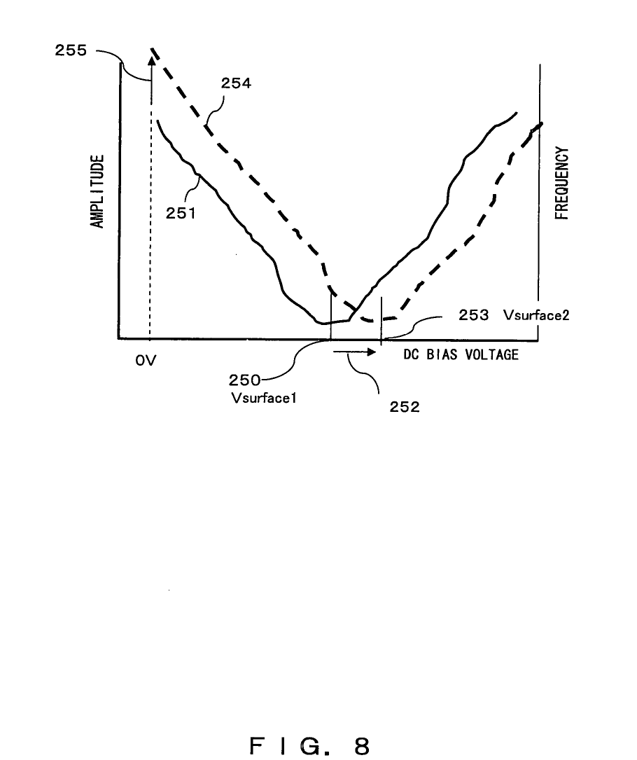

FIG. 8 is a diagram showing the result of a DC bias application test performed using a cMUT, which includes a dielectric film with a large surface potential, according to the present embodiment. The curve 251 shows a DC bias voltage dependency curve (in the case of a membrane surface potential being minus 1000 volts) of a peak frequency on an ultrasound wave side. The point 250 shows a voltage (=V_{surface1}) at which the amplitude along the curve 251 is at the minimum. The curve 254 shows a DC bias voltage dependency curve (in the case of a membrane surface potential being minus 150 volts) of a peak frequency on an ultrasound wave side. The point 253 shows a voltage (=V_{surface2}) at which the amplitude along the curve 254 is at the minimum. The arrow 252 shows an increase of V_{surface}. The arrow 255 shows an increase of a reception signal amplitude at zero (“0”) volt.

Based on this result, the character-V characteristic (i.e., the curves 251 and 254) in terms of a DC bias voltage is confirmed. From FIG. 8, the large reception signal voltage gain when a DC bias voltage is zero (“0”) volt is confirmed. A DC bias voltage corresponding to the valley of the character-V characteristic (the curve 251) is applicable to a surface potential V_{surface} of a dielectric film and does not function as a cMUT when the V_{surface} is smaller than 50 volts. If the V_{surface} is equal to or greater than 50 volts, however, the V-curve shifts from the curve 251 to the curve 254 as the V_{surface} increases. As a result, the maximum amplitude of a reception signal forcee DC bias voltage at zero (“0”) volt becomes large, causing increased sensitivity and an S/N ratio, hence it is favorable. In FIG. 7, the configurations indicating a surface potential at saturation being 50 volts or greater are SiO₂ (the curve 32), three-layer film (the curve 31) consisting of SiO₂, SiNₓ, and Sio₂, and the two-layer film (the curve 31) consisting of SiO₂ and SiNₓ, with all of these films being applied by a heat treatment after forming the film and again after a charging process.

Using the above described configuration, it is possible to obtain a reception signal with a large amplitude by charging a dielectric film even if the DC bias is zero (“0”) volt. The effect of this is that the amplitude at the DC bias voltage at zero volt increases with the surface potential.

Therefore, the use of a dielectric film with a surface potential for a cMUT component makes it possible to perform the same function as a case of applying a DC bias, thereby enabling the cMUT to be driven only by an RF signal without applying a DC bias.

### Second Embodiment

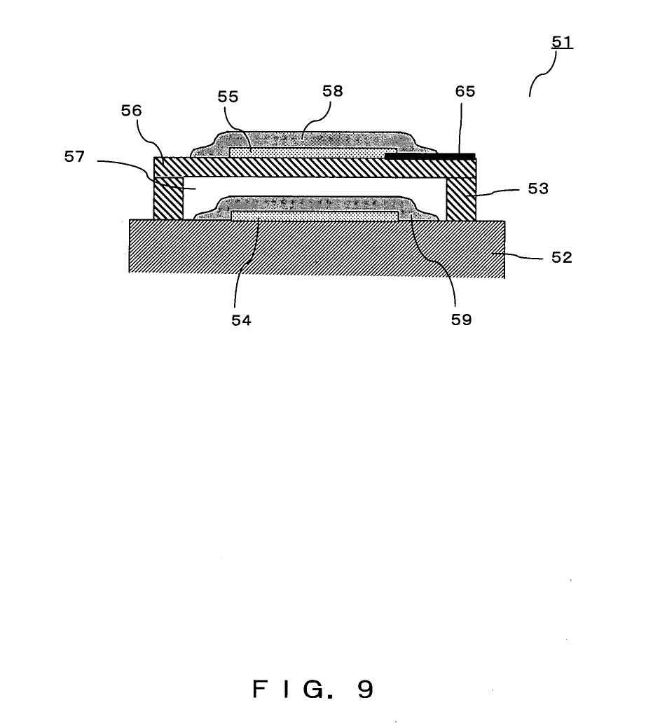

FIG. 9 is an overall cross-section diagram of a cMUT cell's fundamental structure according to the present embodiment. The cMUT cell comprises a silicon substrate 52, dielectric films 58 and 59, a bottom electrode 54, membrane support parts 53, a membrane 56, an upper electrode 55, and a wiring film 65, with cMUT 51 comprising a plurality of the cMUT cells. The differences between this embodiment and the first embodiment are the placement of the bottom electrode 54 on the upper surface of the silicon substrate 52 followed by forming the dielectric film 59, and forming the dielectric film 58 over the upper electrode 55. In this configuration, the Via hole equipment is not required because the bottom electrode 54 contacts with the silicon substrate 52. The numeral 57 is a cavity. Incidentally, the membrane 56 is constituted using a plurality of membrane films in terms of the production process, the same as in the first embodiment.

The above described configuration makes it possible to obtain a more stable effect (i.e., a state corresponding to a DC bias voltage being applied) than the first embodiment.

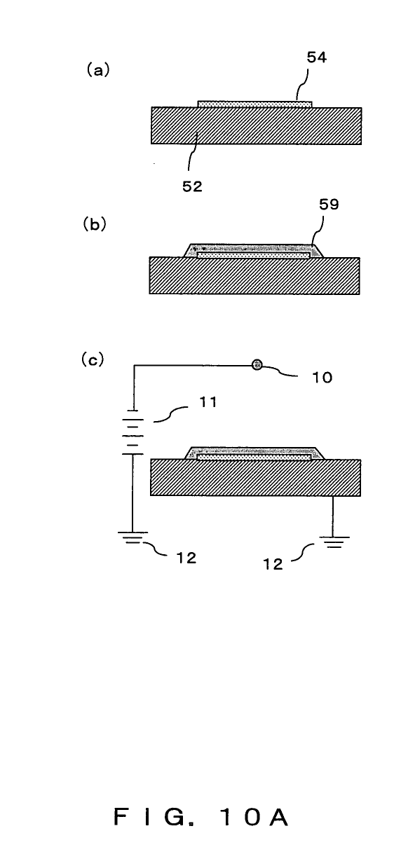

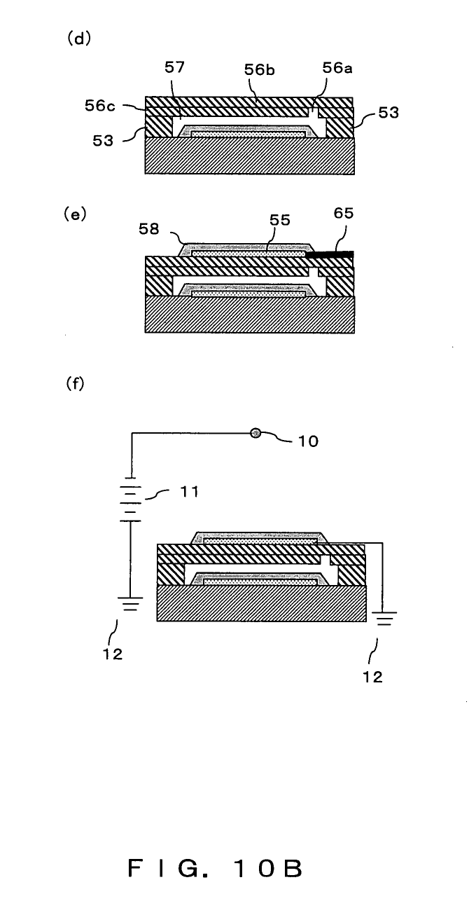

FIG. 10, i.e., FIGS. 10A and 10B, show a cMUT 51 production process according to the present embodiment. The first process forms the bottom electrode 54 made of a thermal resistant metal such as platinum on the low resistance silicon substrate 52 (refer to FIG. 10A (a)), followed by forming a silicon dioxide film (SiO₂ film) 59 by means of rf magnetron sputtering, plasma CVD, vacuum arc plasma method, sol-gel method, et cetera, on the bottom electrode 54. Then, the process applies a heat treatment in the air or a nitrogen environment of a temperature between 300 and 800° C. (refer to FIG. 10A (b)).

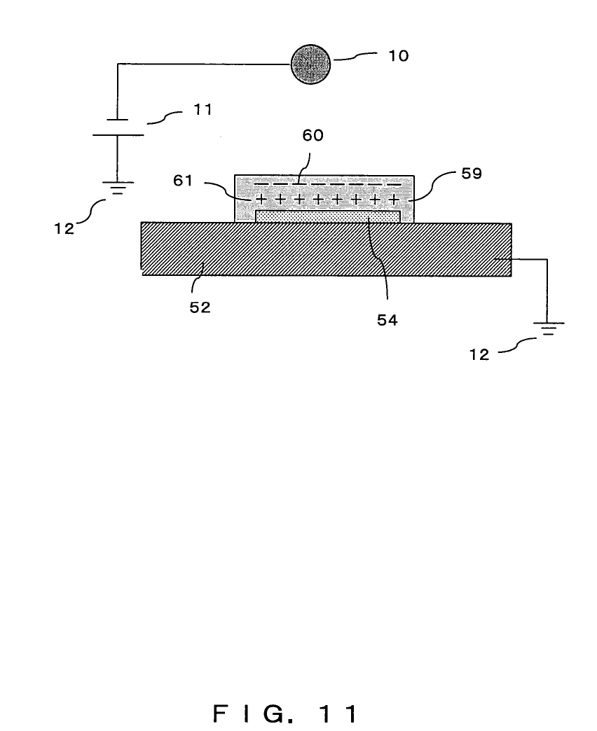

The next process connects the silicon substrate 52 to the ground (the numeral 12) and applies a high voltage DC voltage 11 of several kilovolts between the silicon substrate 52 and a wire form electrode 10, making a corona-discharge and causing the silicon dioxide film charge itself (i.e., an “electretization” process). This charges the front surface of the film with a minus charge (refer to FIG. 10A (c) and FIG. 11), which is described in detail by referring to FIG. 11.

FIG. 11 is a diagram describing a corona discharge according to the present embodiment. In the configuration shown by FIG. 11, a wire form electrode 10 extends in the vertical direction relative to the drawing. The minus side of the high voltage DC voltage 11 is connected to the electrode 10, while the plus side is grounded (at the numeral 12). The electrode 10 is placed above the silicon substrate 52 on which the silicon dioxide film is formed.

In this event, the application of a high voltage DC voltage 11 of several kilovolts causes a corona discharge that makes the electrode 10 discharge a negative charge. This thereby charges the front surface of the silicon dioxide film 59 with a negative charge (the numeral 60) and the side of the silicon substrate with a positive charge (the numeral 61). The corona discharge treatment may be applied while reciprocating the substrate side in a lateral direction in order to obtain an even charge. Alternatively, a grid electrode may be placed between the electrode and a charging treatment target, thereby improving a stability of the corona discharge condition. The description of FIG. 10 is continued.

The next step is an aging treatment to stabilize a charged state of the charged silicon dioxide film 59, e.g., a heat treatment for one hour at 150° C. in the air (refer to FIG. 10A (c)). The stability of a charge over time is important, and therefore the above described heat treatment and the aging treatment after the charge process are indispensable for stabilization (refer to FIG. 7).

The next process forms a cavity 57 (refer to FIG. 10B (d)), for which carried out are: a support part (SiNₓ film) forming, a sacrifice layer poly-silicon film forming, a membrane film (SiNₓ film) forming, a sacrifice layer etching, and an etching hole cover layer forming.

First, themembrane support parts (i.e., SiNₓ film) 53 (e.g., Si₃N₄ film) are formed (refer to FIG. 10B (d)), followed by the formation of the membrane 56c made of Si₃N₄ and the air cavity 57 by sacrifice layer etching, et cetera. The numeral 56c and 56a are a membrane and a membrane hole, respectively, which are required for a sacrifice layer etching process. Then a second membrane film covering the membrane hole 56a is formed, the process of which is the same as the one shown in FIG. 4B.

Then the upper electrode 5 and wiring film 15 made of gold (Au), aluminum (Al), et cetera, are formed (refer to FIG. 10B (e)) . On these, a dielectric film is formed comprising SiO₂ using the rf magnetron sputtering, plasma CVD, vacuum arc plasma, et cetera, followed further by applying a corona charging treatment (i.e., an “electretization” process) (refer to FIG. 10B (f)). Here, a corona charging treatment is applied in the same manner as shown in FIG. 10A (c), with the upper electrode 55 connected to the ground (the numeral 12). Then it is followed by applying an aging process, e.g., a heat treatment for one hour at 150° C. in the air.

The dielectric film may use a silicon nitride film (SiNₓ), or it is best if a layered film made of SiO₂ and Si₃N₄ is used (refer to FIG. 6), in lieu of being limited by a silicon dioxide film. Alternatively, a dielectric film may use a material with a high dielectric constant such as barium titanate BaTiO₃, strontium titanate SrTiO₃, barium-strontium titanate, tantrum penta-oxide, niobium oxide-stabilized tantrum penta-oxide, aluminum nitride, titanium dioxide TiO₂, et cetera.

### Third Embodiment

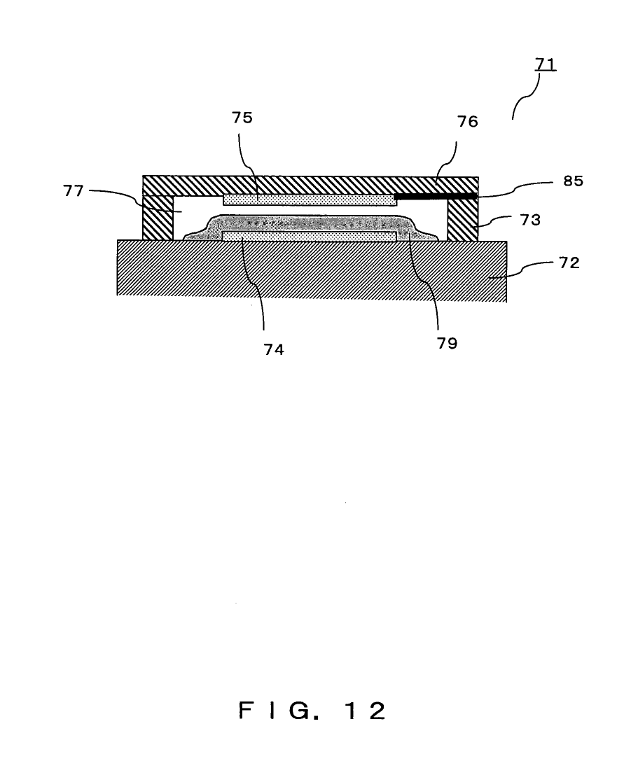

FIG. 12 is an overall cross-section diagram of a cMUT cell's fundamental structure according to the present embodiment. The cMUT cell comprises a silicon substrate 72, a dielectric film 79, a bottom electrode 74, membrane support parts 73, a membrane 76, an upper electrode 75, and a wiring film 85. The cMUT 71 is comprised of a plurality of the cMUT cells. Incidentally, the numeral 77 is a cavity. The differences from the first embodiment and this embodiment are the placement of the bottom electrode 74 on the upper surface of the silicon substrate 72 followed by forming the dielectric film 79, and forming the upper electrode 75 on the lower surface of the membrane 76 (i.e., the surface on the air cavity 77 side). In this configuration, the Via hole equipment is not required because the bottom electrode 74 contacts with the silicon substrate 72.

The above described configuration makes it possible to obtain a more stable effect (i.e., a state corresponding to a DC bias voltage being applied) than the first embodiment.

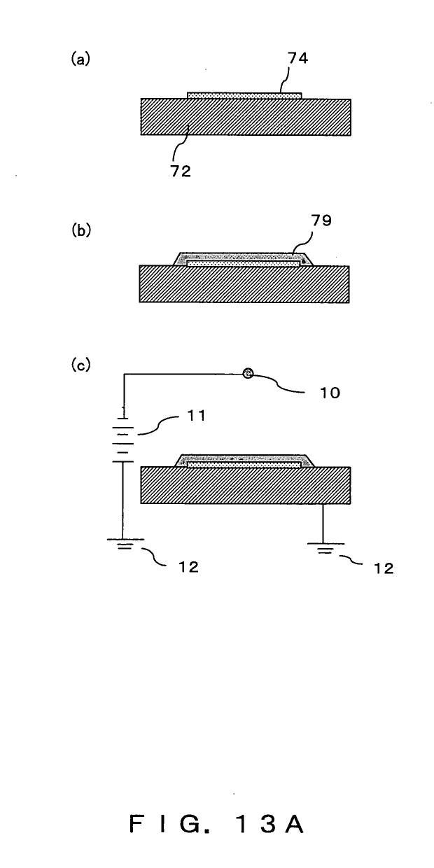

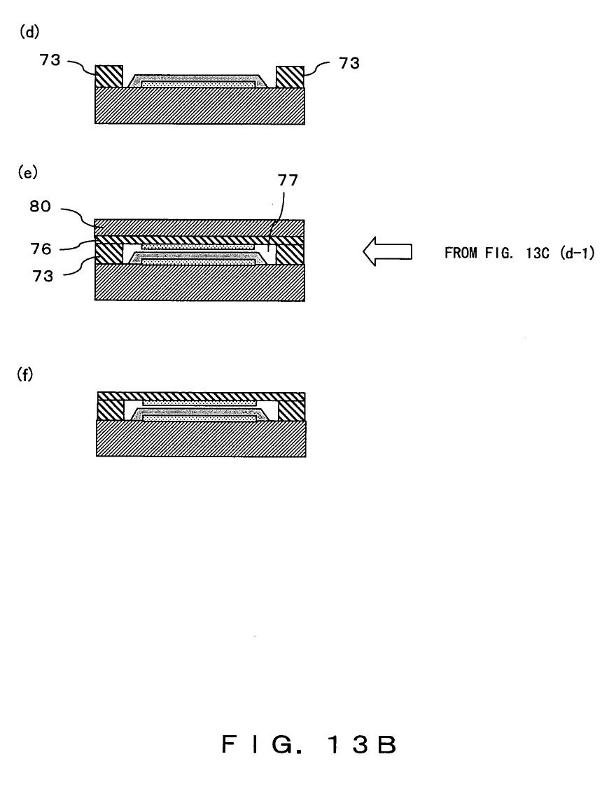

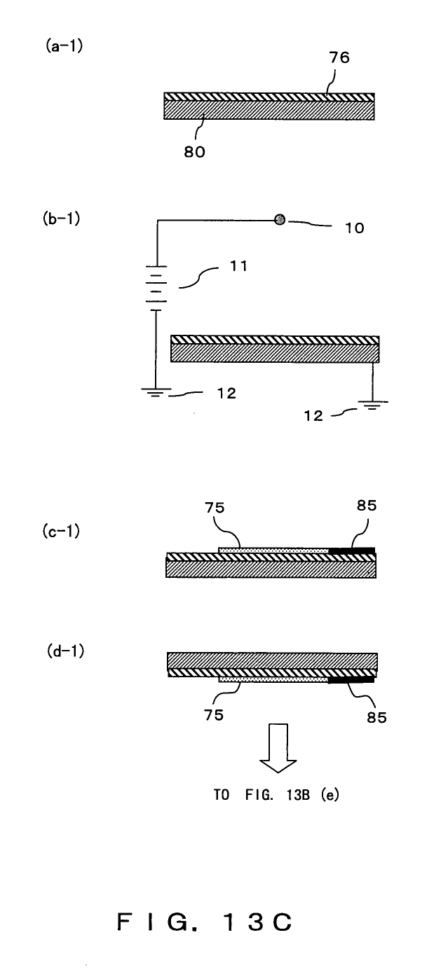

FIG. 13, i.e., FIGS. 13A, 13B and 13C, show a cMUT 71's production process according to the present embodiment. FIGS. 13A (a) through (c) are the same as the second embodiment (FIG. 10A (a) through (c)).

The above described process is followed by forming the membrane support parts 73, comprising an insulator film made of Si₃N₄, et cetera, through film forming such as rf magnetron sputtering (refer to FIG. 13B (d)).

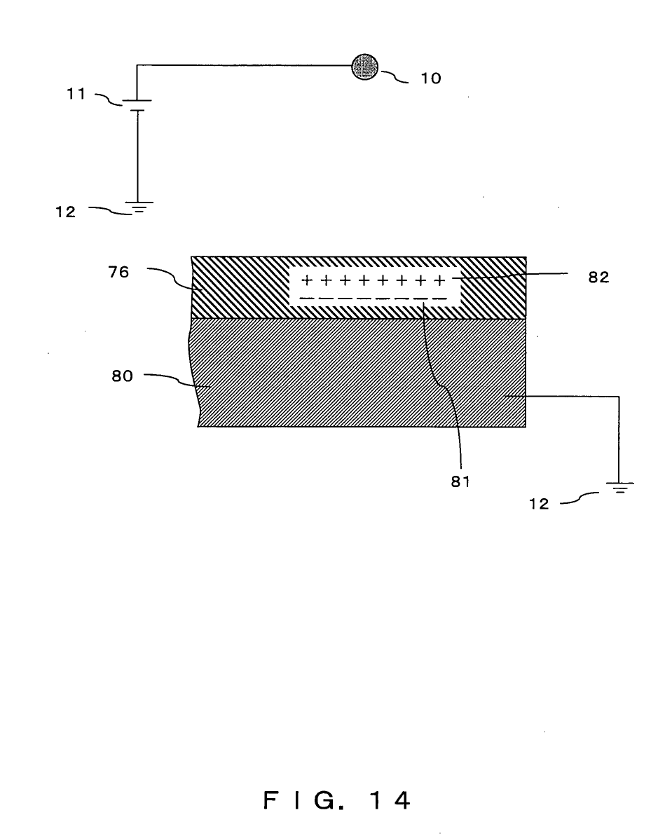

Then, what is shown by FIG. 13C (which is called a structure B) is generated using a process independent of the above described process diagrams of FIG. 13(a) through (d) (the one generated in FIG. 13(a) through (d) is called a structure A). First, the process forms a dielectric film with a high dielectric constant, e.g., a silicon nitride film 76, on a front surface of a silicon substrate 80, which is independent of the structure A, by thermal oxidization, rf magnetron sputtering, plasma CVD, vacuum arc plasma method, sol-gel method, et cetera (refer to FIG. 13C (a-1). This membrane film 76 is heat-treated at a temperature between 300 and 800° C., followed by forming a surface charge using a charging treatment from a corona discharge system (i.e., an “electretization” treatment) (refer to FIG. 13C (b-1)). Here, it is desirable to charge, using a positive charge, the front surface of the dielectric film 76 (i.e., the upper surface of FIG. 13C (b-1)) that has a high dielectric constant. Therefore the polarity of the corona discharge voltage is the opposite polarity (refer to FIG. 14). This is explained in detail by referring to FIG. 14.

FIG. 14 is a diagram describing a corona discharge according to the present embodiment. In FIG. 14, a wire form electrode 10 extends vertically to the drawing. Apositive side of a high voltage DC voltage 11 is connected to the electrode 10, while the negative side is grounded at the numeral 12. The electrode 10 is placed above the silicon substrate 80 on which the dielectric film 76 with a high dielectric constant is formed.

Then, a corona discharge is caused by an application of a high voltage DC voltage 11. Accordingly, a positive charge is discharged from the electrode 10, thereby charging the front surface of the dielectric film 76 having a high dielectric constant with a positive charge (the numeral 82), while the silicon substrate side is charged with a negative charge (the numeral 81). The reason for applying a reverse voltage as compared to FIG. 13A (c) is for increasing the field strength as described in the first embodiment. As shown later, the structure B is turned over in FIG. 13C (d-1) and connected to the structure A. Therefore it is charged in the direction of increasing the field strength when it is turned over. In order to obtain and even charging, the corona discharge treatment may be applied while making the silicon substrate side reciprocate in the lateral direction of the drawing. Alternatively, a grid electrode may be placed between the electrode and a charging treatment target, thereby improving a stability of the corona discharge condition. The description of FIG. 13C is continued below.

An aging treatment is applied to stabilize the charged state of the charged silicon nitride film 76, e.g., a heat treatment for one hour in the air at 150° C. (refer to FIG. 13C (b-1)) . Maintaining the stability of a charge over time is important; therefore the above described heat treatment and aging treatment after the charge process are indispensable for charge stabilization (refer to FIG. 7).

The upper electrode 75 and wiring film 85, which are made of gold (Au), aluminum (Al), et cetera, are formed on the dielectric film 76 with a high dielectric constant (refer to FIG. 13C (c-1)). Here, the completed structure B is turned over (refer to FIG. 13C (d-1) and connected to the structure A, which has been generated in the above described separate process, thereby forming a cavity 77 (refer to FIG. 13B (e)) . Furthermore, the etching application, that has the silicon dioxide film 76's surface including an end point by using a silicon etching fluid such as potassium hydroxide (KOH), forms a membrane consisting of the silicon dioxide film 76 and upper electrode 75 (refer to FIG. 13B (f).

The dielectric film may use a metallic compound film other than silicon, and it is best if the film uses a double-layer film constituted by SiO₂ and Si₃N₄, in lieu of being limited by a silicon nitride film. Alternatively, a dielectric film may use a material with a high dielectric constant, such as barium titanate BaTiO₃, strontium titanate SrTiO₃, barium-strontium titanate, tantrum penta-oxide, niobium oxide-stabilized tantrum penta-oxide, aluminum oxide, titanium dioxide TiO₂, et cetera.

### Fourth Embodiment

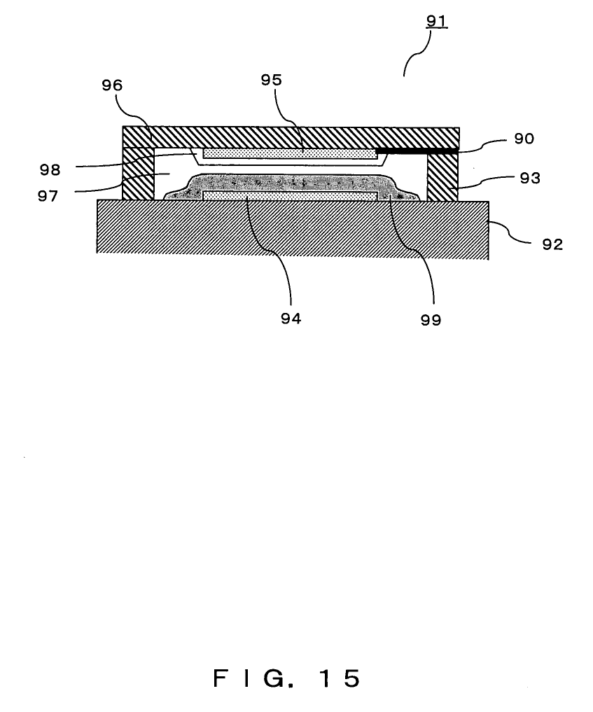

FIG. 15 is an overall cross-section diagram of a cMUT cell's fundamental structure according to the present embodiment. The cMUT cell comprises a silicon substrate 92, dielectric films 98 and 99, a bottom electrode 94, membrane support parts 93, a membrane 96, an upper electrode 95, and a wiring film 90. The cMUT 91 comprises of a plurality of the cMUT cells. Incidentally, the air cavity is represented by 97. The difference between the third embodiment and this embodiment is that the dielectric film 98 covers the surface of the upper electrode 95.

The above described configuration makes it possible to obtain a more stable effect (i.e., a state corresponding to a DC bias voltage being applied) than the first embodiment.

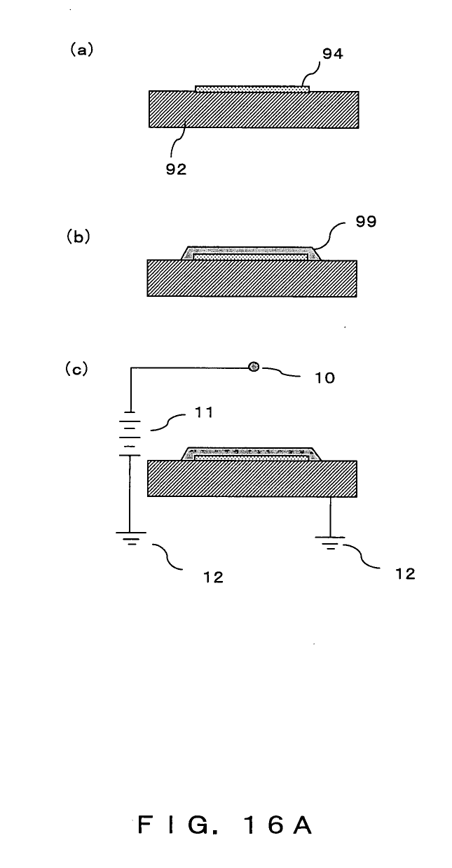

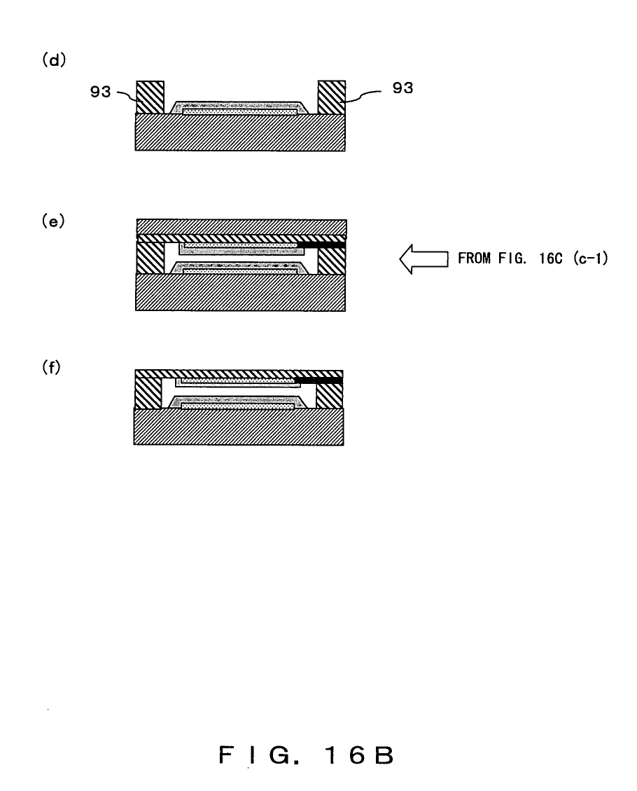

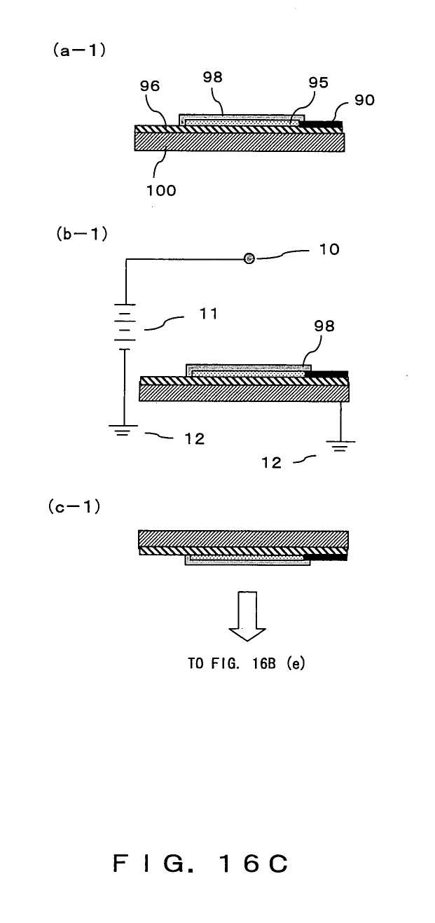

FIG. 16 (FIGS. 16A, 16B and 16C) shows a cMUT 91's production process according to the fourth embodiment. The difference between the third embodiment and the current embodiment is that the structure B (refer to FIG. 16C), prepared by another process, forms a dielectric film 96 on a silicon substrate 100. It also forms the upper electrode 95 and wiring film 90, followed by forming another dielectric film 98 (e.g., a dielectric film having a high dielectric constant such as barium titanate BaTiO₃, strontium titanate SrTiO₃, barium-strontium titanate, tantrum penta-oxide, niobium oxide-stabilized tantrum penta-oxide, aluminum oxide, titanium dioxide TiO₂, et cetera) and applying a heat treatment (refer to FIG. 16C (a-1)).

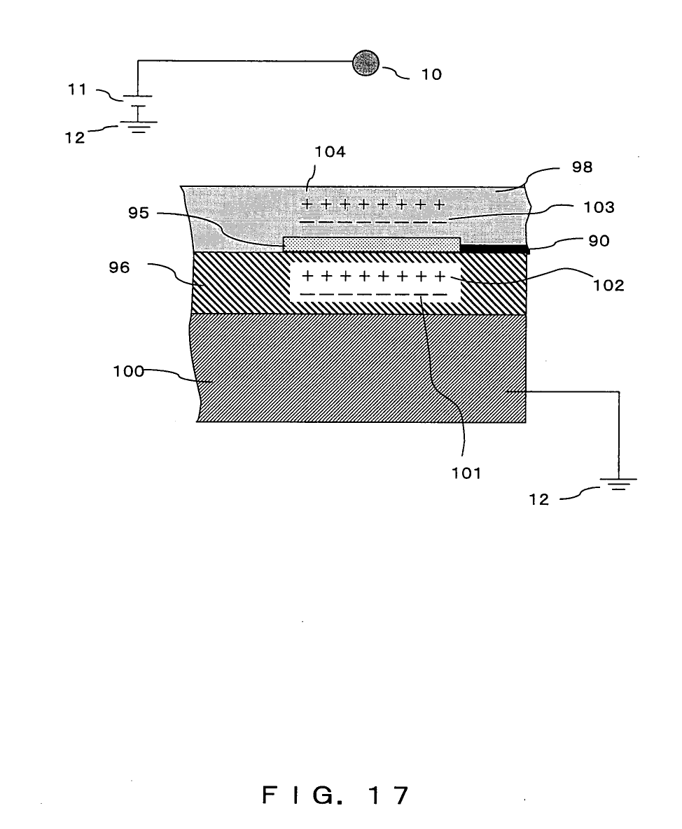

Then, the silicon dioxide film 96 and dielectric film 98 with a high dielectric constant are at once treated for a charging treatment to have surface potentials, respectively, by means of corona discharge system, et cetera (i.e., an “electretization” treatment) (refer to FIG. 16C (b-1). In this case, it is desirable to charge, using a positive charge, the front surface of the dielectric film 98 (i.e., the upper surface of FIG. 16C (b-1)) with a high dielectric constant. Therefore the polarity of the corona discharge voltage is the reversed polarity (refer to FIG. 17). This is explained in detail by referring to FIG. 17.

FIG. 17 is a diagram describing a corona discharge according to the present embodiment. In FIG. 17, a wire form electrode 10 extends in a vertical direction relative to the drawing. The positive side of a high voltage DC voltage 11 is connected to the electrode 10, while the negative side is grounded at the numeral 12. The electrode 10 is placed above the silicon substrate 100 on which the silicon dioxide film 96 and dielectric film 98 are formed.

The application of a high voltage DC voltage 11 of several kilovolts for causing a corona discharge makes the electrode 10 discharge a positive charge. This charges the front surface of the dielectric film 98 with a positive charge (the numeral 104) and the upper electrode 95 side with a negative charge (the numeral 103). Meanwhile, the upper electrode 95 side of the dielectric film 96 is charged with a positive charge (the numeral 102) and the silicon substrate side is charged with a negative charge; both of which are induced by the negative charge on the upper electrode 95 side of the dielectric film 98. By so doing, the silicon dioxide film 96 and dielectric film 98 can be charged at once. What follows the process is the same as that of the third embodiment.

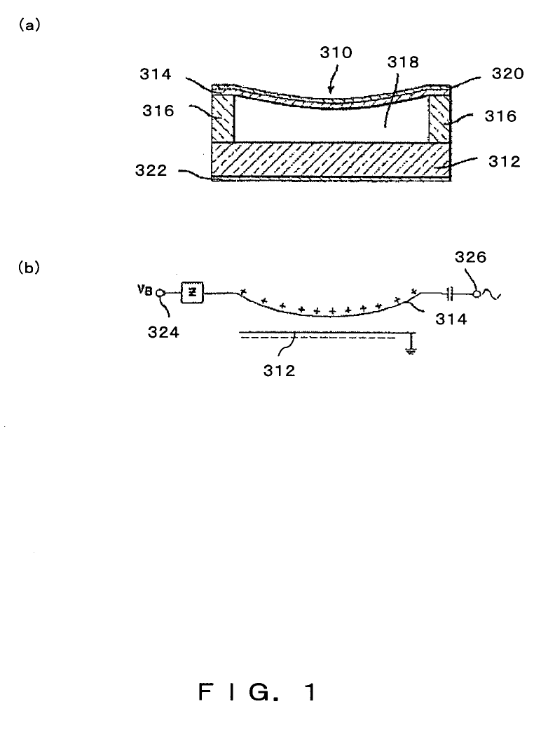

The dielectric film 98, with a high dielectric constant, does not necessarily require charging using the “electretization” treatment. Only the silicon dioxide film 96 may be charged thereby. Contrarily, the silicon dioxide film 96 does not necessarily require charging using the “electretization” treatment. Only the dielectric film 98 with a high dielectric constant may be treated thereby. This occursb ecause only forming the dielectric film 98 with a high dielectric constant or the silicon dioxide film 96 can increase the effect of a charge. The former case requires the process of FIG. 13C (c-1), followed by that of FIG. 16 (a-1), and then that of FIG. 16 (c-1) relating to producing the structure B. The latter case requires the process of FIG. 13C (c-1), followed by forming the dielectric film 98 with a high dielectric constant, and then the process of FIG. 16C (c-1) and thereafter. Meanwhile, maintaining the stability of a charge over time is important; therefore the above described heat treatment and an aging treatment after a charge process is indispensable for stabilization (refer to FIG. 7).

The dielectric film may use a silicon nitride film, or it is best if a layered film made of SiO₂ and Si₃N₄ is used (refer to FIG. 6), in lieu of being limited by a silicon dioxide film. Alternatively, a dielectric film may use a material having a high dielectric constant such as barium titanate BaTiO₃, strontium titanate SrTiO₃, barium-strontium titanate, tantrum penta-oxide, niobiumoxide-stabilized tantrum penta-oxide, aluminum oxide, or titanium dioxide TiO₂.

### Fifth Embodiment

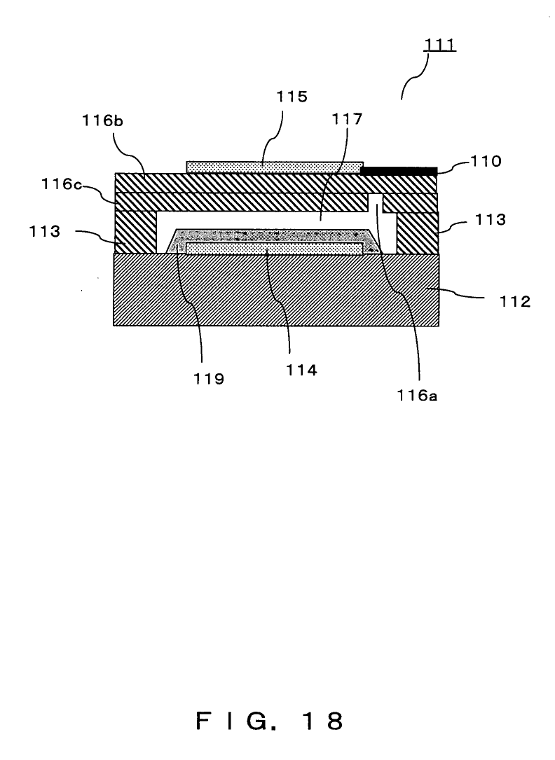

FIG. 18 is an overall cross-section diagram of a cMUT cell's fundamental structure according to the present embodiment. The cMUT cell comprises a silicon substrate 112, a dielectric film 119, a bottom electrode 114, membrane support parts 113, a membrane 116 (including a sacrifice layer material ejection hole 116a, membranes 116b, and 116c), an upper electrode 115, and a wiring film 110. The cMUT 111 is comprised of a plurality of the cMUT cells. Incidentally, a cavity is represented by 117.

The above described configuration makes it possible to obtain a more stable effect (i.e., a state corresponding to a DC bias voltage being applied) than the first embodiment.

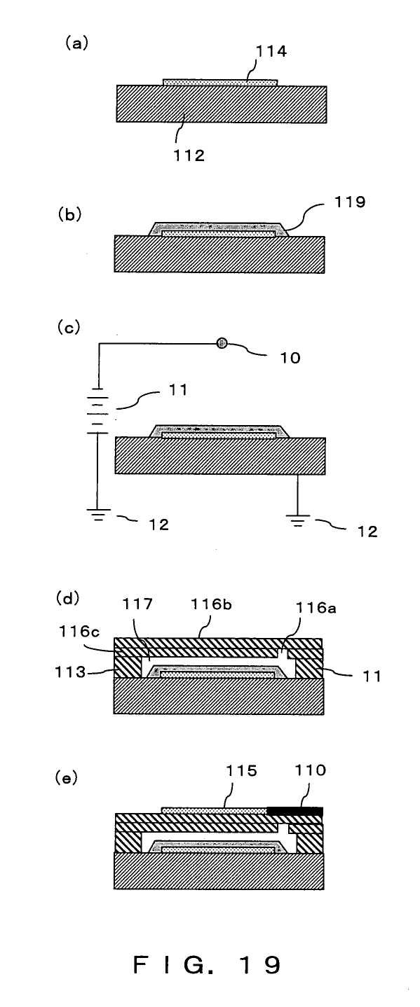

FIG. 19 shows the cMUT 111's production process according to the present embodiment. The first process forms the bottom electrode 114 made of gold (Au), platinum (Pt), et cetera, on the low resistance silicon substrate 112 (refer to FIG. 19(a)). Then the process forms the dielectric film 119, such as silicon dioxide (SiO₂), by using thermal oxidization, rf magnetron sputtering, plasma CVD, vacuum arc plasma method, sol-gel method, et cetera; and by applying a heat treatment in the air or a nitrogen environment at a temperature between 300 and 1000° C. (refer to FIG. 19 (b)).

Then, the silicon substrate 112 is grounded (the numeral 12), and a high voltage DC voltage 11 of several kilovolts is applied between the silicon substrate 112 and a wire form electrode 10 for causing a corona discharge and charging the silicon dioxide film (i.e., an “electretization” process) (refer to FIG. 19(c)). The front surface of the film is charged with a negative charge (refer to FIG. 5). The corona discharging process may be carried out while the substrate side is reciprocated in the lateral direction relative to the drawing for obtaining an even charging. Alternatively, a grid electrode may be placed between the electrode and a charging treatment target, thereby improving a stability of the corona discharge condition. Then, an aging treatment is applied so as to stabilize the charged state, that is, a heat treatment for one hour in the air at 150° C. for example.

The next process forms a cavity 117 (refer to FIG. 19 (d)), that forms a support part (SiNₓ film), a sacrifice layer poly-silicon film, a membrane film (SiNₓ film), a sacrifice layer etching, and an etching hole cover layer.

The first step is to form membrane support parts (e.g., Si₃N₄ film) (refer to FIG. 19(d)), followed by forming the membrane 116c made of Si₃N₄ and the air cavity 117 using a sacrifice layer etching, et cetera. Note that the numeral 116a is a hole diffusing a sacrifice layer material using the sacrifice layer etching, while the membrane 116b is a layer for closing the hole 116a. The process is the same as FIG. 4B.

This is followed by forming the upper electrode 115 and wiring film 110, which are made of gold (Au), aluminum (Al), et cetera (refer to FIG. 19(e)).

Note that the dielectric film may use a silicon nitride film, or it is best if a layered film of SiO₂ and Si₃N₄ is used (refer to FIG. 6), in lieu of being limited by a silicon dioxide film. Meanwhile, maintaining the stability of a charge over time is important; therefore the above described heat treatment and the aging treatment after the charge process are indispensable for stabilization (refer to FIG. 7).

### Sixth Embodiment

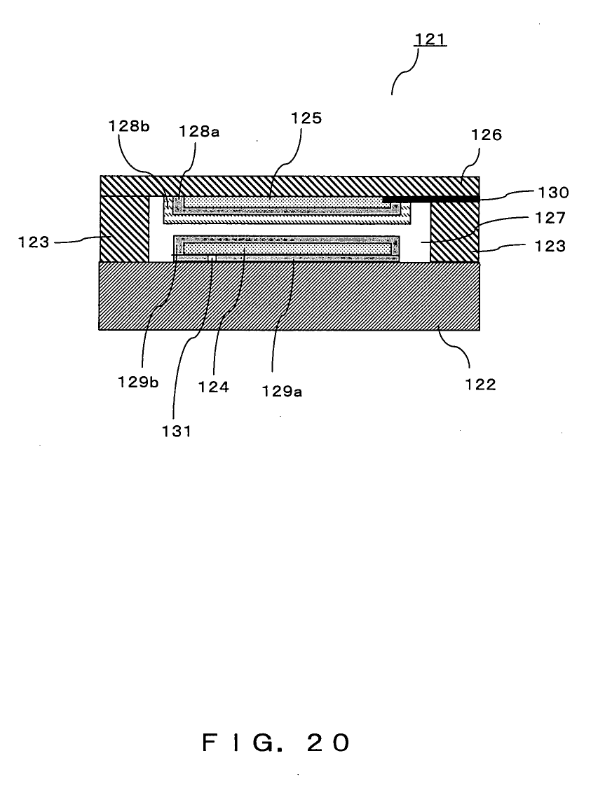

FIG. 20 shows an overall cross-section diagram of a cMUT cell's fundamental structure according to the present embodiment. The cMUT cell comprises a silicon substrate 122, a silicon nitride film 128a, a dielectric film 128b having a high dielectric constant, dielectric films 129a and 129b, a bottom electrode 124, membrane support parts 123, a membrane 126 (including a sacrifice layer material ejection hole 126a, membranes 126b and 126c), an upper electrode 125, and a wiring film 130. The cMUT 121 is comprised of a plurality of the cMUT cells. Incidentally, a cavity is represented by 127. The differences between the fourth embodiment and the current embodiment are the configurations of installing a dielectric film between the bottom electrode 124 and silicon substrate 122, and of further covering the dielectric film covering the upper electrode 125.

The above described configuration makes it possible to obtain a more stable effect (i.e., a state corresponding to a DC bias voltage being applied) than the first embodiment.

The production process of A is almost the same as that of the structure A according to the fourth embodiment (except that the process for forming a dielectric film between the bottom electrode 124 and silicon substrate 122 is added. Specifically, the dielectric film is formed in FIG. 16A (a), followed by forming the bottom electrode), while the production process of the structure B is different. That is, in FIG. 16C, the silicon dioxide film 126 (corresponding to the numeral 96) is formed on the silicon substrate (corresponding to the silicon substrate 100). And form the upper electrode 125 (corresponding to the numeral 95) and wiring film 130 (corresponding to the numeral 90) are formed on the silicon dioxide film 126. Afterward, the silicon nitride film 128a (corresponding to the numeral 98) is formed, and further forming the dielectric film 128b having a high dielectric constant thereon.

The silicon substrate (corresponding to the silicon substrate 100) is then grounded (the numeral 12), a high voltage DC voltage is applied to a wire form electrode placed on the side of the dielectric film 128b having a high dielectric constant, and the front surface thereof is charged by a corona discharge system (which is corresponding to FIG. 16C (b-1)).

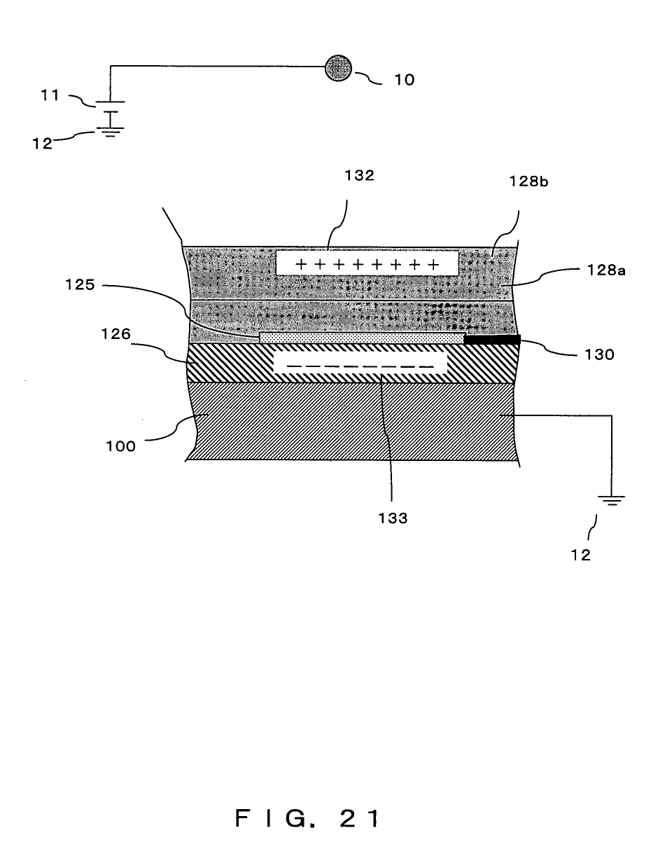

FIG. 21 shows a surface charge whose charge polarity is different from a surface charge formed by charging the dielectric film 129b, which is formed on the bottom electrode of the structure A. This is followed by connecting the structure B obtained by the above described process to the structure A with the former being turned upside down. The continuing processes after the connection are the same as the fourth embodiment.

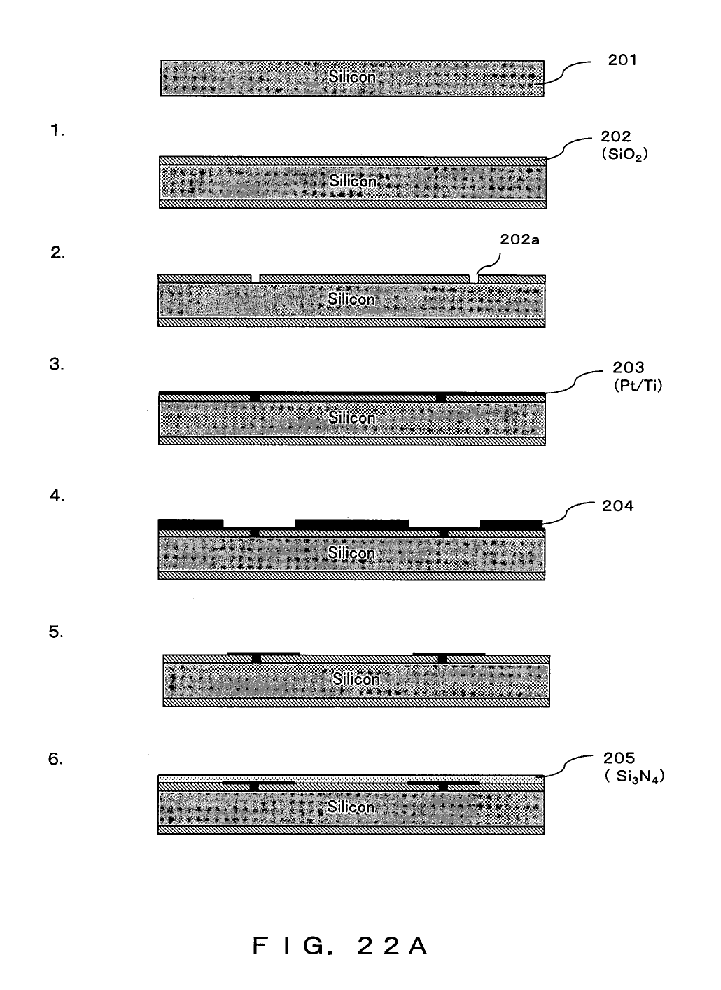

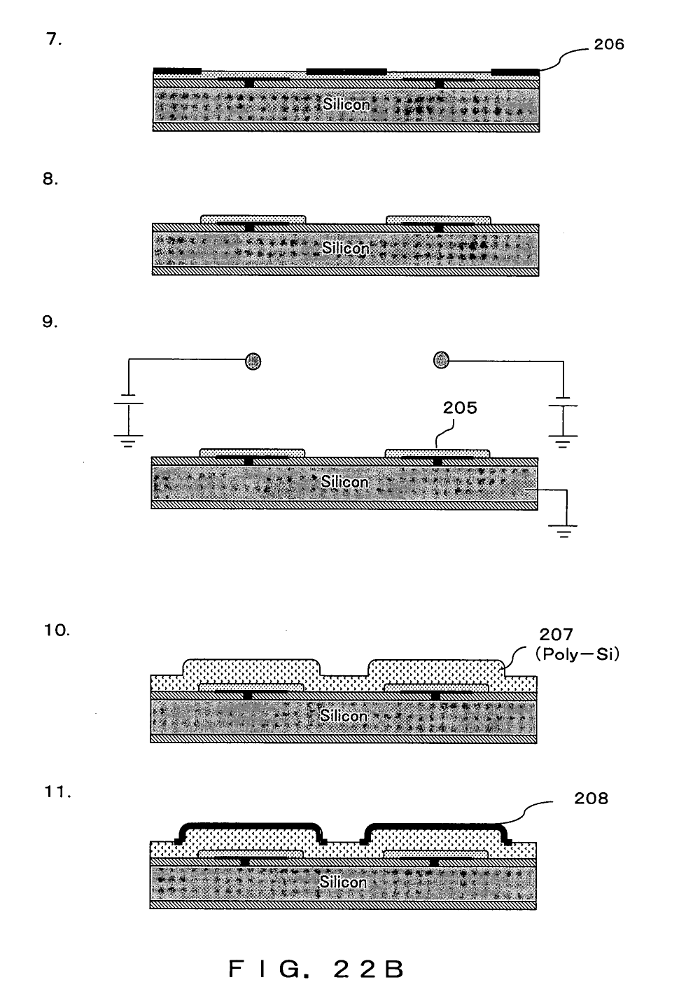

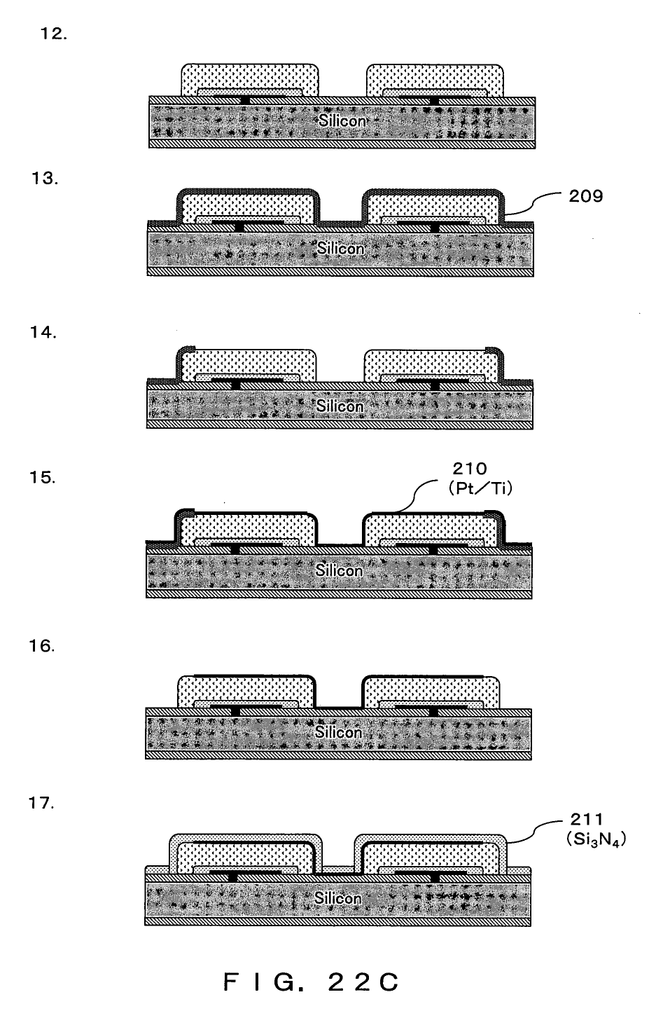

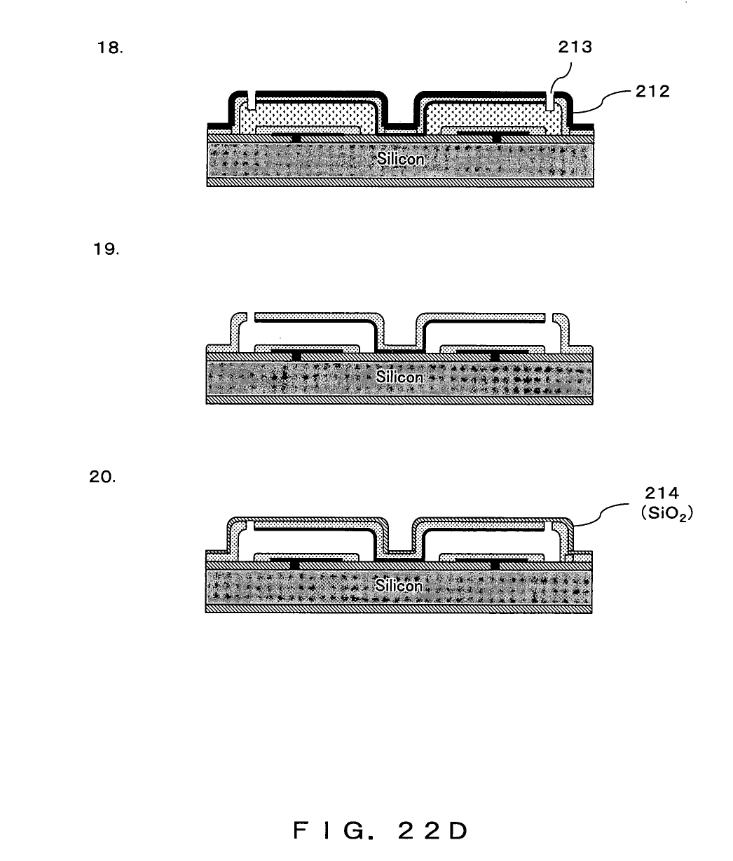

The next description is of the cMUT's production process according to the present embodiment by referring to FIG. 22, i.e., FIGS. 22A, 22B, 22C and 22D.

First, silicon dioxide films (SiO₂) 202 are formed on the front and back surfaces of a silicon substrate 201 (step 1), followed by featuring Via holes 202a (step 2). Then, an electrode 203 made of platinum (Pt)/titanium (Ti) is film-formed by sputtering (step 3). A patterning is then provided by applying resist 204 (e.g., a photo resist material) on the film-formed electrode surface (step 4). Then, an etching is applied for removing Pt/Ti where the resist has not been applied, followed by removing the resist 204 (step 5). Thus the bottom electrode is generated.

Then a film is formed by SiNₓ (e.g., Si₃N₄) 205 (step 6), followed by providing a patterning by applying resist 206 on the film-formed SiNₓ 205 (step 7). The patterning is provided so that the resist 206 is not applied over the bottom electrode 203. Then, an etching is applied for removing the SiNₓ where the resist is not applied, followed by removing the resist 206 (step 8). Thus the bottom electrode surface is covered with SiNₓ.

A heat treatment, a corona discharge (evenly charged across the entire surface by moving the substrate side in the lateral direction of the drawing), and an aging are then applied (step 9). These are the same process as the above described embodiments. This charges the SiNₓ 205. Then a poly-silicon 207 is film-formed (step 10). The poly-silicon 207 is film-formed so that the parts where the bottom electrodes exist swell. Then a patterning process is performed (step 11). In the patterning process, resist 208 is applied on the parts the poly-silicon 207 has been applied in a swelling manner in the step 10.

Next, an etching is conducted for removing the poly-silicon 207 where a resist is not applied, followed by removing the resist 208 (step 12). Then, resist 209 is applied (step 13), followed by a patterning to leave the resist 209 with only both parts of the poly-silicon 207 (step 14).

An electrode 210 is film-formed with Pt/Ti using a sputtering (step 15), followed by removing the resist 209 (step 16). This is further followed using film-forming by SiNₓ (e.g., Si₃N₄) 211 (step 17).

Resist 212 is then applied, and a patterning is applied and etching are carried out in order to feature a sacrifice layer diffusion hole 213 for externally ejecting the sacrifice layer 207 (i.e., poly-silicon) (step 18). Then an etching (e.g., an etching by an ICP-RIE system) is applied for removing the sacrifice layer 207 (i.e., poly-silicon) from the sacrifice layer diffusion hole 213, followed by removing the resist 212 (step 19). The sacrifice layer diffusion hole 213 is sealed by a film-forming 214 with SiO₂ (step 20). Finally, a corona discharge and an aging treatment are applied for charging the SiNₓ film 211 and SiO₂ film 214.

Use of the present invention makes it possible to obtain the same effect as applying a DC bias voltage. Therefore, a cMUT according to the present invention can be driven only by an RF signal or a superimposition of a DC pulse at transmission, without applying a DC bias voltage.

What is claimed is:

1. A capacitive micromachined ultrasonic transducer (cMUT) at least including a silicone substrate, a bottom electrode mounted onto the silicone substrate, a upper electrode mounted facing the bottom electrode and apart therefrom by a predetermined cavity, and a membrane supporting the upper electrode, wherein
one or more layers of dielectric film are formed proximity to at least either one of the bottom electrode and upper electrode, at least one layer of the dielectric film is so to have a surface potential, and the dielectric film having the surface potential is mounted between the silicone substrate and the bottom electrode.

2. The cMUT according to claim 1, wherein
said dielectric film comprises two or more layers.

3. The cMUT according to claim 2, wherein
the surface potential's polarity treated for said dielectric film is the same direction for each of the dielectric films constituting the aforementioned cMUT.

4. The cMUT according to claim 1, wherein
an absolute value of the surface potential of said dielectric film is 50 volts or higher at saturation.

5. The cMUT according to claim 1, wherein
said dielectric film with said surface potential is so formed as to contact with at least either a surface of said bottom electrode or that of said upper electrode.

6. The cMUT according to claim 1, wherein
said membrane is further treated so as to have a surface potential.

7. The cMUT according to claim 6, wherein
said upper electrode is mounted onto a surface on a side facing said bottom electrode among surfaces of said membrane.

8. The cMUT according to claim 2, wherein
said dielectric film is constituted of two layers, that is, an SiO₂ film and an Si₃N₄ film.

9. The cMUT according to claim 2, wherein
said dielectric film is constituted of three layers, that is, an SiO₂ film, an Si₃N₄ film and an SiO₂ film.

10. The cMUT according to claim 1, wherein
said upper electrode is covered with a dielectric film with a high dielectric constant.

11. A production method for a capacitive micromachined ultrasonic transducer (cMUT) at least including a silicone substrate, a bottom electrode mounted onto the silicone substrate, a upper electrode mounted facing the bottom electrode and apart therefrom by a predetermined cavity, and a membrane supporting the upper electrode, comprising the processes for:
forming a dielectric film on the silicone substrate;
applying a corona charging process, by grounding the bottom electrode, so that the dielectric film has a surface potential;
forming the bottom electrode on the dielectric film following the application of the corona charging process thereto;
forming the membrane and a mounting part for supporting the membrane; and
forming the upper electrode on the membrane.

12. A production method for a capacitive micromachined ultrasonic transducer (cMUT) at least including a silicone substrate, a bottom electrode mounted onto the silicone substrate, a upper electrode mounted facing the bottom electrode and apart therefrom by a predetermined cavity, and a membrane supporting the upper electrode, comprising:
a process for forming the bottom electrode on the silicone substrate;
a bottom dielectric film forming process for forming a dielectric film on a surface of the bottom electrode;
a bottom charging process for applying a corona charging process, by grounding the bottom electrode, so that the dielectric film formed by the bottom dielectric film forming process has a surface potential;
a process for forming the membrane and a support part for supporting the membrane;
a process for forming the upper electrode on the membrane;
aupper dielectric film forming process for forming a dielectric film on the upper electrode; and
a upper charging process for applying a corona charging process, by grounding the upper electrode, so that the dielectric film formed by the upper dielectric film forming process has a surface potential.

13. A production method for a capacitive micromachined ultrasonic transducer (cMUT) at least including a silicone substrate, a bottom electrode mounted onto the silicone substrate, a upper electrode mounted facing the bottom electrode and apart therefrom by a predetermined cavity, and a membrane supporting the upper electrode, comprising:
a bottom structural body generation process for generating a bottom structural body by
a process for forming the bottom electrode on a bottom silicone substrates,
a bottom dielectric film forming process for forming a dielectric film on a surface of the bottom electrode,
a bottom charging process for applying a corona charging process, by grounding the bottom electrode, so that a dielectric film formed by the bottom dielectric film forming process has a surface potential, and
a process for forming a support part in order to support the membrane;
a upper structural body generation process for generating a upper structural body by
a upper charging process for applying a corona charging process, by grounding a upper silicone substrate whose surface has been applied by an oxidization treatment, so that the oxidized film on the surface has a surface potential, and
a process for forming the upper electrode on the oxidized film with a surface potential by the upper charging process; and
a process for connecting the bottom structural body generated by the bottom structural body generation process to the upper structural body generated by the upper structural body generation process.

14. A production method for a capacitive micromachined ultrasonic transducer (cMUT) at least including a silicone substrate, a bottom electrode mounted onto the silicone substrate, a upper electrode mounted facing the bottom electrode and apart therefrom by a predetermined cavity, and a membrane supporting the upper electrode, comprising:
a bottom structural body generation process for generating a bottom structural body by
a process for forming a bottom electrode on a bottom silicone substrates,
a bottom dielectric film forming process for forming a dielectric film on a surface of the bottom electrode,
a bottom charging process for applying a corona charging process, by grounding the bottom electrode, so that a dielectric film formed by the bottom dielectric film forming process has a surface potential, and
a process for forming a support part in order to support the membrane;
a upper structural body generation process for generating a upper structural body by
a upper charging process for applying a corona charging process, by grounding a upper silicone substrate whose surface has been applied by an oxidization treatment, so that the oxidized film on the surface has a surface potential,
a process for forming the upper electrode on the oxidized film having a surface potential by the upper charging treatment,
a process for forming a dielectric film having a high dielectric constant on a surface of the upper electrode, and
a third charging process for applying a corona charging process, with the upper electrode being grounded, so that the dielectric film having a high dielectric constant has a surface potential; and
a process for connecting the bottom structural body generated by the bottom structural body generation process to the upper structural body generated by the upper structural body generation process.

15. The production method for a cMUT according to claim 11, wherein
said dielectric film is formed by an rf magnetron sputtering, a plasma CVD (chemical vapor deposition), or a vacuum arc plasma.

16. The production method for cMUT according to claim 11, wherein
a heat treatment is applied after forming said dielectric film.

17. The production method for a capacitive cMUT according to claim 11, including
a process for aging after said corona charging processing.

18. An ultrasonic endoscope apparatus comprising a cMUT according to claim 1.

19. An ultrasonic endoscope apparatus comprising a cMUT produced by a production method noted by claim 11.

20. The production method for a cMUT according to claim 12, wherein
said dielectric film is formed by an rf magnetron sputtering, a plasma CVD (chemical vapor deposition), or a vacuum arc plasma.

21. The production method for a cMUT according to claim 12, wherein
a heat treatment is applied after forming said dielectric film.

22. The production method for a cMUT according to claim 12, including
a process for aging after said corona charging processing.

23. An ultrasonic endoscope apparatus comprising a cMUT produced by a production method noted by claim 12.

24. The production method for a cMUT according to claim 13, wherein
said dielectric film is formed by an rf magnetron sputtering, a plasma CVD (chemical vapor deposition), or a vacuum arc plasma.

25. The production method for a cMUT according to either one of claim 13, wherein
a heat treatment is applied after forming said dielectric film.

26. The production method for a cMUT according to claim 13, including
a process for aging after said corona charging processing.

27. An ultrasonic endoscope apparatus comprising a cMUT produced by a production method noted by claim 13.

28. The production method for a cMUT according to claim 14, wherein
said dielectric film is formed by an rf magnetron sputtering, a plasma CVD (chemical vapor deposition), or a vacuum arc plasma.
